(19) United States
(12) Patent Application Publication
(10) Pub. No.: US 2019/0192413 A1
(43) Pub. Date: Jun. 27, 2019

(54) SKIN ANTI-AGING COMPOSITION CONTAINING DEHYDROGENATED ABIETIC ACID AND COMPOUND K

(71) Applicant: AMOREPACIFIC CORPORATION, Seoul (KR)

(72) Inventors: Young-Gyu KANG, Yongin-si, Gyeonggi-do (KR); Nok Hyun PARK, Yongin-si, Gyeonggi-do (KR); Jun Seong PARK, Yongin-si, Gyeonggi-do (KR)

(21) Appl. No.: 16/331,452

(22) PCT Filed: Sep. 6, 2017
(86) PCT No.: PCT/KR2017/009767

§ 371 Date: Mar. 7, 2019

(30) Foreign Application Priority Data
Sep. 8, 2016 (KR) 10-2016-0115548

(51) Int. Cl. A61K 8/63; A23L 33/10; A61Q 19/08; A61K 8/36; A61K 31/704; A61K 31/192; A61P 17/18

(52) U.S. Cl. CPC A61K 8/63; A23L 33/10; A61Q 19/08; A23V 2002/00; A61K 31/704; A61K 31/192; A61P 17/18; A61K 8/36

(57) ABSTRACT

The present specification provides a composition for preventing skin aging, containing, as active ingredients: dehydrogenated abietic acid, a stereoisomer thereof, a salt thereof, a hydrate thereof, or a solvate thereof; and compound K represented by chemical formula (2), a stereoisomer thereof, a salt thereof, a hydrate thereof, or a solvate thereof, thereby having excellent skin cell proliferation effects, collagen production promotion effects, and Lipofuscin accumulation inhibition effects, while not causing any skin side effects.

### FIELD OF THE INVENTION

The present application relates to a composition for anti-aging of a skin comprising dehydroabietic acid and compound K as active ingredients.

### BACKGROUND OF THE INVENTION

The skin is a primary defense barrier of the human body, which functions to protect various organs in the body from changes in temperature and humidity, and external environmental stimuli such as UV and pollutants, and plays an important role in maintaining homeostasis in vivo. However, excessive physical and chemical stimuli from the external environment, stresses, nutritional deficiency and the like deteriorate the normal function of the skin and accelerate skin aging phenomena such as firmness loss, keratinization, wrinkle formation and the like. To prevent such phenomena and to keep the skin healthier and more elastic, there have been efforts to use cosmetics containing physiologically active substances obtained from various plants, microorganisms and the like, so as to maintain the intrinsic function of the skin and activate skin cells, thus effectively inhibiting skin aging.

However, existing cosmetic raw materials have various problems in that they mostly have insufficient effects or cause side effects on the skin.

### CITATION LIST

### Patent Literature

Patent Literature 1: Korean Patent No. 10-1349248

Patent Literature 2: Korean Patent Laid-Open No. 10-2014-0003198

Patent Literature 3: Korean Patent Laid-Open No. 10-2006-0104161

Patent Literature 4: Korean Patent No. 10-0882780

Patent Literature 5: Korean Patent No. 10-1401658

Patent Literature 6: Korean Patent No. 10-1140039

### Technical Problem

In order to solve the problems, the present invention provides a composition for preventing skin aging, which contains, as active ingredients, dehydroabietic acid and compound K, and thus which has an excellent effect of promoting skin cell proliferation and collagen production and inhibiting lipofuscin accumulation without causing side effects on the skin.

In one aspect, an objective of the present invention is to provide a composition for anti-aging of the skin comprising, as active ingredients, dehydroabietic acid, a stereoisomer thereof, a salt thereof, a hydrate thereof or a solvate thereof; and compound K, a stereoisomer thereof, a salt thereof, a hydrate thereof or a solvate thereof.

In one aspect, an objective of the present invention is to provide a composition for reducing skin wrinkles comprising, as active ingredients, dehydroabietic acid, a stereoisomer thereof, a salt thereof, a hydrate thereof or a solvate thereof; and compound K, a stereoisomer thereof, a salt thereof, a hydrate thereof or a solvate thereof.

In one aspect, an objective of the present invention is to provide a composition for enhancing skin elasticity comprising, as active ingredients, dehydroabietic acid, a stereoisomer thereof, a salt thereof, a hydrate thereof or a solvate thereof; and compound K, a stereoisomer thereof, a salt thereof, a hydrate thereof or a solvate thereof.

### Technical Solution

In one aspect, the present invention provides a composition for anti-aging of the skin comprising, as active ingredients, dehydroabietic acid, a stereoisomer thereof, a salt thereof, a hydrate thereof or a solvate thereof; and compound K, a stereoisomer thereof, a salt thereof, a hydrate thereof or a solvate thereof.

In one aspect of the present invention, a weight ratio of dehydroabietic acid, a stereoisomer thereof, a salt thereof, a hydrate thereof or a solvate thereof; and compound K, a stereoisomer thereof, a salt thereof, a hydrate thereof or a solvate thereof in the composition is 1:1 to 15.

In one aspect of the present invention, the weight ratio is 1:5 to 10.

In one aspect of the present invention, a content of the active ingredients in the composition is 0.0001 to 10 wt % based on the total weight of the composition.

In one aspect of the present invention, the composition is a composition for reducing skin wrinkles.

In one aspect of the present invention, the composition is a composition for enhancing skin elasticity.

In one aspect of the present invention, the composition is a composition for anti-aging of the skin that prevents skin aging by proliferating fibroblasts.

In one aspect of the present invention, the composition is a composition for anti-aging of the skin that prevents skin aging by promoting collagen biosynthesis.

In one aspect of the present invention, the composition is for a composition for anti-aging of the skin that prevents skin aging by inhibiting lipofuscin accumulation in the skin.

In one aspect of the present invention, the composition is a cosmetic composition.

In one aspect of the present invention, the composition is a food composition.

In one aspect of the present invention, the composition is a pharmaceutical composition.

### Effects of the Invention

The composition according to one aspect of the present invention exhibits an excellent effect of reducing skin aging.

The composition according to one aspect of the present invention exhibits an excellent effect of inhibiting skin aging.

The composition according to one aspect of the present invention exhibits an excellent anti-aging effect on the skin without causing side effects on the skin.

The composition according to one aspect of the present invention exhibits an excellent effect of proliferating skin cells without causing side effects on the skin.

The composition according to one aspect of the present invention exhibits an excellent effect of reducing skin wrinkles without causing side effects on the skin.

The composition according to one aspect of the present invention exhibits an excellent effect of enhancing skin elasticity without causing side effects on the skin.

The composition according to one aspect of the present invention exhibits an excellent effect of promoting collagen biosynthesis without causing side effects on the skin.

The composition according to one aspect of the present invention exhibits an excellent effect of inhibiting lipofuscin accumulation without causing side effects on the skin.

The composition according to one aspect of the present invention exhibits a superior anti-aging effect on the skin compared to each of dehydroabietic acid and compound K.

### DETAILED DESCRIPTION OF THE PREFERRED EMBODIMENTS

Hereinafter, the present invention will be described in further detail.

One aspect of the present invention is to provide a composition for preventing skin aging, which comprises, as active ingredients, dehydroabietic acid and compound K, and thus which has an excellent effect of promoting skin cell proliferation and collagen production and inhibiting lipofuscin accumulation without causing side effects on the skin. In order to achieve the above objective, one aspect of the present invention provides a composition for anti-aging of the skin comprising, as active ingredients, dehydroabietic acid represented by Formula 1 below and compound K represented by Formula 2 below.

Dehydroabietic acid as described herein may be represented by the following Formula 1:

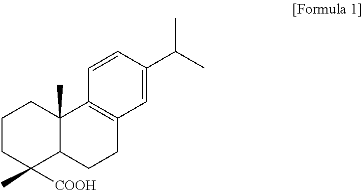 [SMILES: CC(C)c1ccc2c(c1)CCC1[C@](C)(C(=O)O)CCC[C@]21C]

Compound K as described herein may be represented by the following Formula 2:

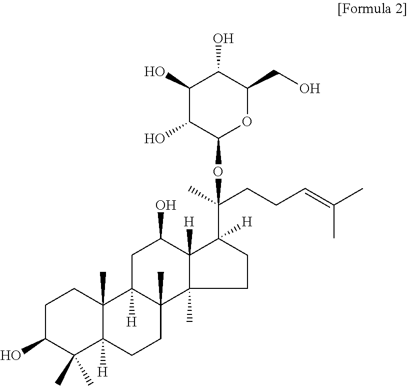

One aspect of the present invention provides a composition for anti-aging of the skin comprising, as active ingredients, dehydroabietic acid, a stereoisomer thereof, a salt thereof, a hydrate thereof or a solvate thereof and compound K, a stereoisomer thereof, a salt thereof, a hydrate thereof or a solvate thereof.

As used herein, the term “stereoisomer” refers to isomers that have the same molecular formula and sequence of bonded atoms, but differ in the three-dimensional orientations of their atoms in space. It may be a diastereomer or an optical isomer. In addition, the diastereomer may be a geometrical isomer or a diastereomer that is not a geometrical isomer.

As used herein, the term “salt” refers to a “pharmaceutically or cosmetically acceptable salt”. The term “pharmaceutically or cosmetically acceptable” means that which is useful in preparing a pharmaceutical composition that is generally safe, non-toxic and biologically or otherwise desirable and includes that which is acceptable for veterinary use as well as human pharmaceutical and cosmetic uses. As used herein, the term “pharmaceutically or cosmetically acceptable salts” means salts which are pharmaceutically or cosmetically acceptable, as defined above, and which possess the desired pharmacological activity. Such salts include (1) acid addition salts formed with inorganic acids such as hydrochloric acid, hydrobromic acid, sulfuric acid, nitric acid, phosphoric acid, and the like; or with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts that are formed when an acidic proton present in a parent compound is replaced.

As used herein, the term “hydrate” means a “pharmaceutically or cosmetically acceptable hydrate”. The “hydrate” exists when the compound of the present invention contains water. The hydrate may contain one or more water molecules per molecule of the compound of the present invention. Illustrative non-limiting examples include monohydrates, dehydrates, trihydrates and tetrahydrates. The hydrate may contain at least one molecule of compound of the present invention per molecule of water. Illustrative non-limiting examples include semi-hydrates. In an embodiment, the water may be held in the crystal in various ways, and thus the water molecule may occupy lattice positions in the crystal, or may form a bond with a salt of the compound as described herein. The hydrate must be “acceptable” in the sense of not being deleterious to the recipient thereof.

As used herein, the term “solvate” means a “pharmaceutically or cosmetically acceptable solvate”. The term “solvate” means that the compound of the invention contains one or more pharmaceutically acceptable solvents. The solvate may contain one or more molecules of solvent per molecule of compound of the present invention, or may contain one or more molecules of compound of the invention per molecule of solvent. In one embodiment, the solvent may be held in the crystal in various ways, and thus the solvent molecule may occupy lattice positions in the crystal, or may form a bond with a salt of the compound as described herein.

As used herein, “anti-aging of the skin” refers to the ability to slow down, stop or prevent the aging process known in the art. Specifically, it may mean the ability to enhance skin elasticity and reduce wrinkles by proliferating skin cells and skin collagen biosynthesis and inhibiting lipofuscin accumulation, although not limited thereto.

In one aspect of the present invention, the weight ratio of dehydroabietic acid, a stereoisomer thereof, a salt thereof, a hydrate thereof or a solvate thereof; and compound K, a stereoisomer thereof, a salt thereof, a hydrate thereof or a solvate thereof in the composition may be 1:1 to 15. Specifically, the weight ratio may be 1:1 or more, 1:2 or more, 1:3 or more, 1:4 or more, 1:5 or more, 1:5.5 or more, or 1:6 or more. In addition, the weight ratio may be 1:15 or less, 1:14 or less, 1:13 or less, 1:12 or less, 1:11 or less, 1:10 or less, 1:9 or less, 1:8 or less, or 1:7.5 or less. Preferably, the weight ratio of dehydroabietic acid and compound K in the composition may be 1:5 to 10. When the weight ratio of dehydroabietic acid and compound K is within the above range, the effects of proliferating skin cells, promoting collagen biosynthesis and inhibiting lipofuscin accumulation in skin cells are remarkably excellent. In addition, when the weight ratio of dehydroabietic acid:compound K is 1:1 or more, lipofuscin accumulation is further inhibited as compared with the case where the weight ratio is 1:less than 1. In addition, when the weight ratio of dehydroabietic acid:compound K is 1:15 or less, lipofuscin accumulation is further increased as compared with the case where the weight ratio is 1:more than 15.

In one aspect of the present invention, the content of the active ingredients in the composition may be 0.0001 to 10 wt % based on the total weight of the composition. Specifically, the content of the active ingredients may be 0.0001 wt % or more, 0.0005 wt % or more, 0.001 wt % or more, 0.005 wt % or more, 0.01 wt % or more, 0.05 wt % or more, 0.1 wt % or more, 0.5 wt % or more, 1 wt % or more, 2 wt % or more, or 5 wt % or more based on the total weight of the composition. In addition, the content of the active ingredients may be 10 wt % or less, 9 wt % or less, 8 wt % or less, 7 wt % or less, 6 wt % or less, 5 wt % or less, 3 wt % or less, 1 wt % or less, 0.1 wt % or less, 0.01 wt % or less, or 0.001 wt % or less based on the total weight of the composition. If the content of the active ingredients is less than 0.0001 wt %, the effects of anti-aging of skin, skin wrinkle reduction and skin elasticity enhancement will be insignificant. If the content of the active ingredients is more than 10 wt %, an increase in the content will not lead to a significant increase in the effects, and thus will not be cost-effective.

In one aspect of the present invention, the composition may be a composition for reducing skin wrinkles.

In one aspect of the present invention, the composition may be a composition for enhancing skin elasticity.

In one aspect of the present invention, the composition may be a composition for anti-aging of the skin that prevents skin aging by proliferating fibroblasts.

In one aspect of the present invention, the composition may be a composition for anti-aging of the skin that prevents skin aging by promoting collagen biosynthesis.

In one aspect of the present invention, the composition may be a composition for anti-aging of the skin that prevents skin aging by inhibiting lipofuscin accumulation in the skin. Lipofuscin is known to interfere with DNA and RNA synthesis and protein synthesis and destroy cellular enzymes required for important chemical metabolic processes. Thus, the inhibition of lipofuscin is an important factor in anti-aging of the skin.

In one aspect of the present invention, the composition may be a cosmetic composition. The formulation of the cosmetic composition is not specifically limited, and can be suitably selected depending on the intended use. For example, it may be one or more formulations selected from the group consisting of skin lotion, skin softer, skin toner, astringent, lotion, milk lotion, moisture lotion, nourishing lotion, massage cream, nourishing cream, moisture cream, hand cream, foundation, essence, nourishing essence, pack, soap, cleansing foam, cleansing lotion, cleansing cream, body lotion, and body cleanser, although not limited thereto.

When the formulation of the present invention is a paste, a cream or a gel, it may contain, as a carrier ingredient, animal fiber, plant fiber, wax, paraffin, starch, tragacanth, cellulose derivatives, polyethylene glycol, silicone, bentonite, silica, talc, or zinc oxide, etc.

When the formulation of the present invention is a powder or a spray, it may contain, as a carrier ingredient, lactose, talc, silica, aluminum hydroxide, calcium silicate or polyamide powder. Particularly, when the formulation is a spray, it may further contain a propellant such as chlorofluorohydrocarbon, propane/butane or dimethyl ether.

When the formulation of the present invention is a solution or an emulsion, it may contain, as a carrier ingredient, a solvent, a solubilizer or an emulsifier. Examples of this carrier ingredient include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan.

When the formulation of the present invention is a suspension, it may contain, as a carrier ingredient, a liquid diluent such as water, ethanol or propylene glycol, a suspending agent such as ethoxylated isostearyl alcohol, polyoxyethylene sorbitol ester or polyoxyethylene sorbitan ester, microcrystalline cellulose, aluminum metahydroxide, bentonite, agar, or tragacanth, etc.

When the formulation of the present invention is a surfactant-containing cleanser, it may contain, as a carrier ingredient, aliphatic alcohol sulfate, aliphatic alcohol ether sulfate, sulfosuccinic monoester, isethionate, imidazolinium derivatives, methyl taurate, sarcosinate, fatty acid amide ether sulfate, alkyl amidobetaine, aliphatic alcohol, fatty acid glyceride, fatty acid diethanolamide, vegetable oil, lanolin derivatives, or ethoxylated glycerol fatty acid ester.

The content of the active ingredient is not specifically limited, but it may be 0.0001 to 10 wt % based on the total weight of the composition. When the content of the active ingredients is within the above range, the composition can exhibit excellent effects without side effects.

In one aspect of the present invention, the cosmetic composition may further contain, in addition to the active ingredients, a functional additive and other ingredients that are generally used in cosmetic compositions. The functional additive may be selected from the group consisting of water-soluble vitamins, oil-soluble vitamins, polypeptides, polysaccharides, sphingolipids, and seaweed extracts.

In one aspect of the present invention, the cosmetic composition of the present invention may, if necessary, contain, in addition to the functional additive, ingredients that are generally used in cosmetic compositions. Examples of such additional ingredients include oil and fat ingredients, moisturizers, emollients, surfactants, organic and inorganic pigments, organic powder, UV absorbers, preservatives, sterilizers, antioxidants, plant extracts, pH adjusters, alcohols, colorants, fragrance, blood circulation stimulants, skin coolers, deodorants, purified water, etc.

In one aspect of the present invention, the composition may be a composition for external application to the skin.

In one aspect of the present invention, the composition may be a pharmaceutical composition. The pharmaceutical composition according to one aspect of the present invention may be formulated into various oral or parenteral dosage formulations. When the composition is formulated, generally used diluents or excipients such as fillers, extenders, binders, wetting agents, disintegrants, surfactants, etc. are used. Solid preparations for oral administration include tablets, pills, powders, granules, soft or hard capsules, etc., and are prepared by mixing at least one compound with at least one excipient, for example, starch, calcium carbonate, sucrose or lactose, gelatin, etc. Further, lubricants such as magnesium stearate, talc, etc. are used in addition to simple excipients. Liquid preparations for oral administration include suspensions, solutions, emulsions, syrups, etc., and various excipients such as a wetting agent, a sweetener, a flavoring agent, a preservative, etc. as well as water and liquid paraffin, which are generally used simple diluents, may be added. Formulations for parenteral administration include sterile aqueous solutions, non-aqueous solvents, suspensions, emulsions, freeze-dried formulations, and suppositories. Examples of the non-aqueous solvents or the suspensions include propylene glycol, polyethylene glycol, vegetable oil such as olive oil, injectable esters such as ethyloleate, etc. Examples of the matrix for suppositories include witepsol, macrogol, tween 61, cacao butter, laurinum, glycerogelatin, etc.

In one aspect of the present invention, the active ingredients of the pharmaceutical dosage form of the composition of the present invention may be in the form of their pharmaceutically acceptable salts. In addition, the active ingredients of the composition may be administered alone or in combination with other pharmaceutically active compounds or may be used in the form of an appropriate combination. The salt is not specifically limited as long as it is pharmaceutically acceptable. For example, hydrochloric acid, sulfuric acid, nitric acid, phosphoric acid, hydrofluoric acid, hydrobromic acid, formic acid, acetic acid, tartaric acid, lactic acid, citric acid, fumaric acid, maleic acid, succinic acid, methanesulfonic acid, benzenesulfonic acid, toluenesulfonic acid, naphthalenesulfonic acid, etc. may be used.

In one aspect of the present invention, determination on dosage of the active ingredients is within the knowledge of those skilled in the art. The daily dose of the active ingredients may vary depending on various factors, including the severity of the symptom, the time of onset of the symptom, the subject's age and health conditions, complications, etc. For an adult, the composition may generally be administered at a dose of 1 μg/kg to 200 mg/kg, preferably 50 μg/kg to 50 mg/kg, 1-3 times a day. The dose does not limit the scope of the present invention in any way.

The pharmaceutical composition according to one aspect of the present invention may be formulated into any pharmaceutically appropriate form including oral formulations such as powder, granule, tablet, soft or hard capsule, suspension, emulsion, syrup, aerosol, etc., agents for external application to the skin such as ointment, cream, etc., suppository, injection, sterile solution for injection, etc. according to commonly employed methods. Preferably, it may be formulated into an injection or an agent for external application to the skin.

The composition according to one aspect of the present invention may be administered to mammals including rat, mouse, cattle, human, etc. via various routes including parenteral and oral routes. All types of administration may be expected. For example, it may be administered orally, transdermally, rectally, intravenously, intramuscularly, subcutaneously, intrauterinally, or intracerebroventricularly.

The composition according to one aspect the present invention may be administered via various routes that can be easily selected by those skilled in the art. In particular, the pharmaceutical composition according to one aspect of the present invention may be applied on the skin as an agent for external application to the skin.

In one aspect of the present invention, the composition may be a food composition. In one aspect of the present invention, the food composition includes a health food composition. The composition according to one aspect of the present invention may be provided as various types of food additives or functional foods. For example, it may be formulated into fermented milk, cheese, yogurt, juice, probiotics, tablets, granules, drinks, caramels, diet bars, etc. It may be processed into general tea leaves, tea bags, health supplements and the like, or in the form of various other food additives. In one embodiment, the composition may contain other ingredients that can provide synergic effect without negatively affecting the desired main effect, etc. For example, in order to improve the properties, it may further comprise an additive such as flavoring, colorant, sterilizer, antioxidant, preservative, moisturizer, thickener, mineral salt, emulsifier, synthetic polymer, etc. In addition, it may further comprise an auxiliary ingredient such as water-soluble vitamin, oil-soluble vitamin, polypeptide, polysaccharide, seaweed extract, etc. These ingredients may be appropriately selected by those skilled in the art considering the formulation or use. The amount of these ingredients may be determined within a range not negatively affecting the purpose and effect of the present invention. For example, the content of the ingredients may be 0.01-5 wt %, for example, 0.01-3 wt %, based on the total weight of the composition. The composition according to the present invention may be formulated into various forms such as solutions, emulsions, viscous mixtures, tablets, powders and the like. The composition may be administered according to various methods such as simple drinking, injection, spraying or squeezing.

Hereinafter, the present invention will be described in further detail with reference to examples. It will be apparent to those skilled in the art that these examples are for illustrative purposes only and are not intended to limit the scope of the present invention.

### Example 1 and Comparative Examples 1 and 2

The dehydroabietic acid of Formula 1 above purchased from Ramidus AB [symbol] was used in Comparative Example 1, the compound K of Formula 2 above purchased from Ambo Institute [symbol] was used in Comparative Example 2 and a mixture obtained by mixing the dehydroabietic acid and the compound K at a weight ratio of 1:7 was used in Example 1. Test Examples 1 to 3 were conducted for the samples of Comparative Examples 1 and 2 and Example 1.

### Test Example 1: Effect on Skin Cell Proliferation

In order to examine the effect on skin cell proliferation that reduces wrinkles and enhances skin elasticity, human normal fibroblasts were seeded into a 96-well microplate at a density of 1×10⁴ cells/well, and incubated in DMEM (Dulbecco's Modified Eagle's Medium) medium for 24 hours. After incubation, the medium was replaced with serum-free DMEM media containing 10 ppm of each of dehydroabietic acid (Comparative Example 1), compound K (Comparative Example 2), and a mixture of dehydroabietic acid and compound K (weight ratio of 1:7; Example 1), and then the cells were further incubated for 24 hours. Then, 10 μl of 3-(4,5-dimethylthiazol-2-yl)-2,5-diphenyl tetrazolium (MTT, 5 mg/ml) was added to each well, which was then allowed to stand for 4 hours, followed by removal of the medium. Then, 100 μl of dimethyl sulfoxide solution was added to each well, and the absorbance at 570 nm was measured with a microplate reader.

Based on the experiment results, the cell proliferation effect was calculated using the following equation 1. The results of the calculation are shown in Table 1 below.

$$Cell proliferation effect ( \% ) = [ \frac{\begin{matrix}absorbance of group treated with sample - \\ absorbance of control group\end{matrix}}{absorbance of control group} ] \times 100 \qquad [ Equation 1 ]$$

TABLE 1
Cell proliferation effect

| | Absorbance | Cell proliferation |
|---|---|---|
| Ingredient name (concentration) | (570 nm) | effect (%) |
| Control group (0 ppm) | 0.355 | — |
| Comparative Example 1: | 0.546 | 53.8 |
| dehydroabietic acid (10 ppm) | | |
| Comparative Example 2: | 0.520 | 46.5 |
| compound K (10 ppm) | | |
| Example 1: | 0.644 | 81.4 |
| a mixture of dehydroabietic | | |
| acid and compound K | | |
| (1:7) (10 ppm) | | |

As can be seen from the results in Table 1 above, the mixture of dehydroabietic acid and compound K (Example 1) shows a 1.51 times and 1.75 times higher skin cell proliferation effect than the same concentration of dehydroabietic acid (Comparative Example 1) and compound K (Comparative Example 2), respectively.

### Test Example 2: Effect on Promotion of Collagen Biosynthesis

In order to examine the effect on the promotion of collagen biosynthesis that reduces wrinkles and enhances skin elasticity, human normal fibroblasts were seeded into a 96-well microplate at a density of 1×10⁴ cells/well, and incubated in DMEM (Dulbecco's Modified Eagle's Medium) medium for 24 hours.

After incubation, the medium was replaced with serum-free DMEM media containing 10 ppm of each of dehydroabietic acid (Comparative Example 1), compound K (Comparative Example 2), and a mixture of dehydroabietic acid and compound K (weight ratio of 1:7; Example 1) and then the cells were further incubated for 48 hours. At 24 hours before completion of the incubation, 10 μM ascorbic acid as the positive control was added to each well to promote collagen synthesis. After completion, each well was washed, and the medium was replaced with serum-free DMEM medium, after which the cells were further incubated for 24 hours. After incubation, the supernatant of each well was collected, and the amount of procollagen type-I C-peptide (PICP) in the supernatant was measured using a kit (Takara, Kyoto, Japan) to determine the amount of newly synthesized collagen. The amount of PICP that is the amount of collagen synthesized is expressed in the unit of ng/2×10⁴ cells in Table 2 below.

TABLE 2
Effect of promoting collagen biosynthesis

| | Amount of collagen |
|---|---|
| | synthesized |
| Ingredient name (concentration) | (ng/2 × 10⁴) |
| Control group (0 ppm) | 125 |
| Ascorbic acid (10 ppm) | 186 |
| Comparative Example 1: | 174 |
| dehydroabietic acid (10 ppm) | |
| Comparative Example 2: | 162 |
| compound K (10 ppm) | |
| Example 1: | 289 |
| a mixture of dehydroabietic | |
| acid and compound K | |
| (1:7)(10 ppm) | |

As can be seen from the results in Table 2 above, the mixture of dehydroabietic acid and compound K (Example 1) shows a 1.66 times and 1.78 times higher collagen biosynthesis promotion effect than the same concentration of dehydroabietic acid (Comparative Example 1) and compound K (Comparative Example 2), respectively.

### Test Example 3: Effect on Inhibition of Lipofuscin Accumulation in the Skin Cells

In order to examine the effect on the inhibition of lipofuscin accumulation that prevents aging, human normal fibroblasts were seeded into a 96-well microplate at a density of 1×10⁴ cells/well, and incubated in DMEM (Dulbecco's Modified Eagle's Medium) medium for 24 hours.

After incubation, the medium was replaced with serum-free DMEM media containing 10 ppm of each of dehydroabietic acid (Comparative Example 1), compound K (Comparative Example 2), and a mixture of dehydroabietic acid and compound K (weight ratio of 1:7; Example 1). Then, the cells were subcultured in a fresh medium every 48 hours. After 10 passages of subculture, the fluorescence of the self-luminescent protein lipofuscin in the cells was measured using a microplate reader with the excitation wavelength of 360 nm and the emission wavelength of 570 nm.

Based on the experiment results, the effect on inhibition of lipofuscin accumulation was calculated using the following equation 2. The results of the calculation are shown in Table 3 below.

$$Effect of inhibition of lipofuscin accumulation ( \% ) = [ \frac{\begin{matrix}absorbance of control group - \\ absorbance of group treated with sample\end{matrix}}{absorbance of control group} ] \times 100 \qquad [ Equation 2 ]$$

TABLE 3
Effect of inhibition of lipofuscin accumulation

| | Absorbance | Lipofusin accumulation |
|---|---|---|
| Ingredient name (concentration) | (570 nm) | inhibition rate (%) |
| Control group (0 ppm) | 0.650 | — |
| Comparative Example 1: | 0.465 | 28.5 |
| dehydroabietic acid (10 ppm) | | |
| Comparative Example 2: | 0.448 | 31.1 |
| compound K (10 ppm) | | |
| Example 1: | 0.310 | 52.3 |
| a mixture of dehydroabietic | | |
| acid and compound K | | |
| (1:7)(10 ppm) | | |

As can be seen from the results in Table 3 above, the mixture of dehydroabietic acid and compound K (Example 1) shows a 1.83 times and 1.69 times higher inhibitory effect on lipofuscin accumulation than the same concentration of dehydroabietic acid (Comparative Example 1) and compound K (Comparative Example 2), respectively.

Hereinafter, formulation examples of the cosmetic composition having an anti-aging effect on the skin according to one aspect of the present invention will be described. However, it may be formulated into various other forms. These examples are for illustrative purposes only and are not intended to limit the scope of the present invention.

### Formulation Example 1: Skin Toner

Skin toner is prepared according to a conventional method with the composition shown in Table 4 below.

TABLE 4

| Ingredient | Content (wt %) |
|---|---|
| Example 1 | 0.1 |
| Glycerin | 3.0 |
| Butylene glycol | 2.0 |
| Propylene glycol | 2.0 |
| Carboxyvinyl polymer | 0.1 |
| PEG 12 nonylphenyl ether | 0.2 |
| Polysorbate 80 | 0.4 |
| Ethanol | 10.0 |
| Triethanolamine | 0.1 |
| Preservative, colorant, fragrance | q.s. |
| Purified water | Balance |

### Formulation Example 2: Nourishing Cream

Nourishing cream is prepared according to a conventional method with the composition shown in Table 5 below.

TABLE 5

| Ingredient | Content (wt %) |
|---|---|
| Example 1 | 2.0 |
| Polysorbate 60 | 1.5 |
| Sorbitan sesquioleate | 0.5 |
| PEG 60 hydrogenated castor oil | 2.0 |
| Liquid paraffin | 10.0 |
| Squalane | 5.0 |
| Caprylic/capric triglyceride | 5.0 |
| Glycerin | 5.0 |
| Butylene glycol | 3.0 |
| Propylene glycol | 3.0 |
| Triethanolamine | 0.2 |
| Preservative, colorant, fragrance | q.s. |
| Purified water | Balance |

### Formulation Example 3: Massage Cream

Massage cream is prepared according to a conventional method with the composition shown in Table 6 below.

TABLE 6

| Ingredient | Content (wt %) |
|---|---|
| Example 1 | 1.0 |
| Wax | 10.0 |
| Polysorbate 60 | 1.5 |
| PEG 60 hydrogenated castor oil | 2.0 |
| Sorbitan sesquioleate | 0.8 |
| Liquid paraffin | 40.0 |
| Squalane | 5.0 |
| Caprylic/capric triglyceride | 4.0 |
| Glycerin | 5.0 |

### Formulation Example 4: Pack

Pack is prepared according to a conventional method with the composition shown in Table 7 below.

TABLE 7

| Ingredient | Content (wt %) |
|---|---|
| Example 1 | 5.0 |
| Polyvinyl alcohol | 13.00 |
| L-ascorbic acid-2-phosphate magnesium salt | 1.00 |
| Lauroylhydroxyproline | 1.00 |
| Water soluble collagen (1% aqueous solution) | 2.00 |
| 1,3-butylene glycol | 3.00 |
| Ethanol | 5.00 |
| Purified water | Balance |

While the present invention has been particularly shown and described with reference to specific embodiments thereof, those skilled in the art will appreciate that these specific embodiments are merely preferred embodiments and that the scope of the present invention is not limited thereto. Thus, the scope of the present invention is defined by the appended claims and their equivalents.

1. A method for anti-aging of a skin comprising administrating an effective amount of a composition comprising, as active ingredients, dehydroabietic acid, a stereoisomer thereof, a salt thereof, a hydrate thereof or a solvate thereof; and
compound K represented by following Chemical Formula 2, a stereoisomer thereof, a salt thereof, a hydrate thereof or a solvate thereof; to a subject in need thereof.

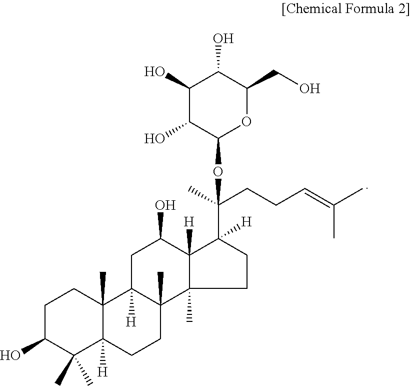

2. The method for anti-aging of the skin according to claim 1,
wherein a weight ratio of dehydroabietic acid, the stereoisomer thereof, the salt thereof, the hydrate thereof or the solvate thereof; and compound K, the stereoisomer thereof, the salt thereof, the hydrate thereof or the solvate thereof in the composition is 1:1 to 15.

3. The method for anti-aging of the skin according to claim 2,
wherein the weight ratio is 1:5 to 10.

4. The method for anti-aging of the skin according to claim 1,
wherein a content of the active ingredients is 0.0001 to 10 wt % based on the total weight of the composition.

5. The method for anti-aging of the skin according to claim 1,
wherein the composition is a composition for reducing skin wrinkles.

6. The method for anti-aging of the skin according to claim 1,
wherein the composition is a composition for enhancing skin elasticity.

7. The method for anti-aging of the skin according to claim 1,
wherein the composition prevents skin aging by proliferating fibroblasts.

8. The method for anti-aging of the skin according to claim 1,
wherein the composition prevents skin aging by promoting collagen biosynthesis.

9. The method for anti-aging of the skin according to claim 1,
wherein the composition prevents skin aging by inhibiting lipofuscin accumulation in the skin.

10. The method for anti-aging of the skin according to claim 1,
wherein the composition is a cosmetic composition.

11. The method for anti-aging of the skin according to claim 1,
wherein the composition is a food composition.

12. The method for anti-aging of the skin according to claim 1,
wherein the composition is a pharmaceutical composition.
